(19) United States
(12) Patent Application Publication
(10) Pub. No.: US 2023/0310587 A1
(43) Pub. Date: Oct. 5, 2023

(54) NOVEL IMMUNOGENIC CORONAVIRUS PEPTIDES AND COMPOSITIONS THEREOF

(71) Applicant: nference,Inc., Cambridge, MA (US)

(72) Inventors: Michiel Niesen, Cambridge, MA (US); Venkataramanan Soundararajan, Andover, MA (US); Rohit Suratekar, Kolhapur (IN); Pritha Ghosh, Kolkata (IN); Karthik Murugadoss, Cambridge, MA (US); Greg Donadio, Cambridge, MA (US); Aiveliagaram Venkatakrishnan, Woburn, MA (US)

(21) Appl. No.: 18/102,363

(22) Filed: Jan. 27, 2023

Related U.S. Application Data
(60) Provisional application No. 63/304,359, filed on Jan. 28, 2022.

(51) Int. Cl. A61K 39/215; A61P 37/04

(52) U.S. Cl. CPC A61K 39/215; A61P 37/04; A61K 2039/53

(57) ABSTRACT

This application relates to isolated proteins comprising an amino acid sequence which comprises at least 2 mutations relative to a SARS-CoV-2 Spike protein template sequence, isolated nucleic acid encoding said proteins, and vaccine compositions comprising said proteins.

### CROSS-REFERENCE TO RELATED APPLICATIONS

This application claims the benefit of U.S. Provisional Application No. 63/304,359, filed Jan. 28, 2022, which is hereby incorporated by reference in its entirety.

### REFERENCE TO A SEQUENCE LISTING XML

This application contains a Sequence Listing which has been submitted electronically in XML format. The Sequence Listing XML is incorporated herein by reference. Said XML file, created on Jun. 2, 2023, is named LBH-02301_SL.xml and is 7,547 in size.

### BACKGROUND

The ongoing COVID-19 pandemic has resulted in SARS-CoV-2 viral infections in over 350 million people worldwide and deaths of over 5.5 million people, in just the last 2 years. 1 Although vaccination against COVID-19 has proven highly effective in preventing infection and severe disease,²⁻¹¹ there is concern about the lasting effectiveness of first-generation vaccines against new viral variants. 12-17 The Spike protein is the main target of current SARS-CoV-2 vaccines, an essential viral protein that is accessible to antibodies. However, mutations in the Spike protein, can alter the binding affinity of antibodies that are induced by current vaccines,^{15,18-20} resulting in a weaker immune response and reduced vaccine effectiveness. Despite existing mRNA technology that can rapidly advance COVID-19 vaccine development after a new variant of concern (VOC) is identified, a deployable vaccine composition can still take over 6 months to materialize. Thus, there is clearly a critical need to move away from reactive vaccine manufacturing towards prediction future viral variants and proactive design of vaccines against said possible future variants. Vaccines that can protect against future SARS-CoV-2 variants will therefore have to be designed by anticipating mutations to the Spike protein that will affect antibody binding.

### SUMMARY

In some aspects of the invention, provided herein is an isolated protein comprising an amino acid sequence selected from Appendix B. Such isolated proteins may comprise at least 2 mutations relative to a SARS-CoV-2 Spike protein template sequence, and said SARS-CoV-2 Spike protein template sequence is selected from Appendix A.

In other aspects of the invention, provided herein is an isolated nucleic acid encoding the isolated protein disclosed herein.

In additional aspects of the invention, provided herein are expression constructs comprising the isolated nucleic acid disclosed herein.

In further aspects of the invention, provided herein is a host cell comprising an expression construct disclosed herein.

In another aspect of the invention, provided herein are methods of producing an isolated protein, said methods comprising expressing an isolated protein disclosed herein and at least partly purifying said isolated protein. Also provided herein are vaccine compositions comprising the isolated proteins disclosed herein and a pharmaceutically acceptable carrier. Also provided herein are vaccine compositions comprising the isolated nucleic acids disclosed herein.

In yet further aspects of the invention, provided herein are methods of preventing or mitigating a SARS-CoV-2 infection in a subject, comprising administering to the subject a vaccine composition disclosed herein.

Unless otherwise defined, all technical and scientific terms used herein have the same meaning as commonly understood by one of ordinary skill in the art to which this disclosure pertains. Preferred methods and materials are described below, although methods and materials similar or equivalent to those described herein can also be used in the practice or testing of the presently disclosed methods and compositions. All publications, patent applications, patents, and other references mentioned herein are incorporated by reference in their entirety.

### BRIEF DESCRIPTION OF THE DRAWINGS

FIG. 1 illustrates the prevalence of variants of concern in the time-windows used for model training and validation in the various validation test cases. (A) Sequences collected early in the pandemic (March 2020 - August 2020), before the emergence of any VOCs. (B) Sequences collected before emergence of the Delta variant (March 2020 - February 2021). (C) Sequences collected before emergence of the Omicron variant (March 2020 - November 2021).

FIG. 2 depicts test case 1; the single-position VOC score for classifying positions based on whether they are mutated in future VOCs (using data from March 2020 - August 2020). (A) ROC curve using the single-position VOC score to classify whether a position will be mutated in a future VOC (Alpha, Beta, Gamma, or Delta). (B) Confusion table, quantifying the rates of true and false predictions obtained when using the optimal classifier threshold obtained in the ROC analysis.

FIG. 3 depicts test case 2; the single-position VOC score used to predict future VOC positions for VOCs not used in classifier training (using data from March 2020 - August 2020). (A) ROC curve using the single-position VOC score to classify whether a position will be mutated in a future VOC (Alpha, Beta, or Gamma). Confusion table, quantifying the rates of true and false predictions obtained when using the optimal classifier threshold obtained in the ROC analysis, for: (B) the training data, (C) the test set of all known Delta VOC positions, and (D) the test set of known Delta VOC positions that do not occur in any other VOC. (E) Summary of the enrichment of confirmed future VOC positions in the predictions, quantified using the positive predictive value and false omission rate.

FIG. 4 depicts test case 3; the single-position VOC score used to predict future VOC positions for VOCs not used in classifier training (using data from March 2020 - February 2021). (A) ROC curve using the single-position VOC score to classify whether a position will be mutated in a future VOC (Alpha, Beta, or Gamma). Confusion table, quantifying the rates of true and false predictions obtained when using the optimal classifier threshold obtained in the ROC analysis, for: (B) the training data, (C) the test set of all known Delta VOC positions, and (D) the test set of known Delta VOC positions that do not occur in any other VOC. (E) Summary of the enrichment of confirmed future VOC positions in the predictions, quantified using the positive predictive value and false omission rate.

FIG. 5 depicts test case 4; the single-position VOC score used to predict future VOC positions for VOCs not used in classifier training (using data from March 2020 - November 2021). (A) ROC curve using the single-position VOC score to classify whether a position will be mutated in a future VOC (Alpha, Beta, Gamma, or Delta). Confusion table, quantifying the rates of true and false predictions obtained when using the optimal classifier threshold obtained in the ROC analysis, for: (B) the training data, (C) the test set of all known Omicron VOC positions, and (D) the test set of known Omicron VOC positions that do not occur in any other VOC. (E) Summary of the enrichment of confirmed future VOC positions in the predictions, quantified using the positive predictive value and false omission rate.

FIG. 6 depicts structural site 1, highlighted on a monomer from the Spike protein structure (PDB ID: 6vsb). This site contains the following predicted future VOC mutations (positions shown as black dots), relative to the reference Spike protein sequence (GenBank: QHD43416.1): L141del/L141F, H245Y, R246del/R246I, and G261V/G261C/G261R/G261D.

FIG. 7 depicts structural site 2, highlighted on a monomer from the Spike protein structure (PDB ID: 6vsb). This site contains the following predicted future VOC mutations (positions shown as black dots), relative to the reference Spike protein sequence (GenBank: QHD43416.1): L176F, I210T, R214L, L216F, and S221L.

FIG. 8 depicts structural site 3, highlighted on a monomer from the Spike protein structure (PDB ID: 6vsb). This site contains the following predicted future VOC mutations (positions shown as black dots), relative to the reference Spike protein sequence (GenBank: QHD43416.1): A520S, A522S/A522V/A522P, E554G/E554Q, and K558N.

FIG. 9 depicts structural site 4, highlighted on a monomer from the Spike protein structure (PDB ID: 6vsb). This site contains the following predicted future VOC mutations (positions shown in as black dots), relative to the reference Spike protein sequence (GenBank: QHD43416.1): Q414K, N439K, R346S/R346I, F490S/F490L, and S494P/S494L.

FIG. 10 depicts structural site 5, highlighted on a monomer from the Spike protein structure (PDB ID: 6vsb). This site contains the following predicted future VOC mutations (positions shown as black dots), relative to the reference Spike protein sequence (GenBank: QHD43416.1): L822F, D936Y/D936N/D936H, L938F, and S939F.

FIG. 11 depicts structural site 6, highlighted on a monomer from the Spike protein structure (PDB ID: 6vsb). This site contains the following predicted future VOC mutations (positions shown as black dots), relative to the reference Spike protein sequence (GenBank: QHD43416.1): T719I, T723I, A1070S/A1070V, Q1071H/Q1071L, and N1074S/N1074D.

FIG. 12 depicts the spike protein reference template (SEQ ID NO: 1), into which the predicted future VOC mutations are introduced.

FIG. 13 depicts the spike protein Delta template (SEQ ID NO: 2), into which the predicted future VOC mutations are introduced. Shown in white are positions that are unchanged from the reference template, shown in black are Delta VOC derived key mutations that are specific to this template.

FIG. 14 depicts the spike protein Omicron template (SEQ ID NO: 3), into which the predicted future VOC mutations are introduced. Shown in white are positions that are unchanged from the reference template, shown in black are Omicron VOC derived key mutations that are specific to this template.

### DETAILED DESCRIPTION

### Identification of Future Variants of Concern

Without being bound by theory, it is proposed herein that future variants of concern (VOC) will be stably mutated at positions in the SARS-CoV-2 genome that have exhibited high mutability in the past. Disclosed herein are proteins whose design are based on the SARS-CoV-2 Spike protein sequence and are intended for future vaccine compositions. Statistical modeling and machine learning techniques were used to first identify specific amino-acid level changes (substitutions and deletions) that were predicted to occur in future variants of concern. Positions with sequence variability are more mutable and were expected to be enriched in future Variant of Concern (VOC) mutations. A classifier was trained that uses sequence variability as a feature to classify viral sequence positions based on whether they are part of future VOCs. The workflow for the prediction of future VOC mutations was validated against historical SARS-CoV-2 mutation data, showing a strong capability in predicting future VOC mutations. Structural and functional constraints were then used to design the relevant immunogenic proteins that form the core of vaccine compositions against the predicted viral variants.

As used herein, the words “a” and “an” can mean one or more than one. As used in the claims in conjunction with the word “comprising,” the words “a” and “an” can mean one or more than one. As used herein, “another” can mean at least a second or more.

As used herein, a “subject” shall mean a vertebrate animal including but not limited to a human, non-human primate, mouse, rat, guinea pig, rabbit, cow, dog, cat, horse, goat, bird, reptile, or fish. In some embodiments of the invention, a subject is a mammal. In some embodiments, the subject may be a domesticated animal, a wild animal, or an agricultural animal. Thus, the invention can be used to inhibit virus particle infectivity and to treat or reduce viral infection in human and non-human subjects. For instance, methods and compositions of the invention can be used in veterinary applications (for examples in zoos, reserves, farms, in the wild, etc.) as well as in human treatment regimens. In some embodiments of the invention, the subject is a human. In some embodiments of the invention, a subject is at risk of having, or has a viral infection.

As used herein, the expression “preventing or mitigating” infection means improving, reducing, or alleviating at least one symptom or biological consequence of virus infection (i.e., SARS-CoV infection) in a subject, and/or reducing or decreasing virus titer, load, replication or proliferation in a subject following exposure to a virus. The expression “preventing or mitigating a SARS-CoV-2 infection” also includes shortening the time period during which a subject exhibits at least one symptom or biological consequence of the infection. Methods for treating virus infection, according to the present invention, comprise administering a pharmaceutical composition of the present invention to a subject after the subject is infected with the virus and/or after the subject exhibits or is diagnosed with one or more symptoms or biological consequences of virus infection.

As will be appreciated by those of skill in the relevant art, the symptom or biological consequence of SARS-CoV infection may include one or more of nasal congestion, sinus congestion, runny nose, sneezing, body (muscle) ache, head ache, chills, fever, cough, sore throat, fatigue, ear ache, or a diagnostic indicator of infection, e.g., detection of SARS-CoV by viral culture, hemagglutinin agglutination inhibition (HAI) assay, immunofluorescence, or nucleic acid-based detection (e.g., RT-PCR) using an appropriate specimen (e.g., nasal swab, nasopharyngeal swab, throat swab, endotracheal aspirate, sputum, bronchial wash, etc.). Thus, a subject who tests positive for infection by a diagnostic assay is considered a subject exhibiting a “symptom or biological consequence” of said virus infection.

In some aspects of the invention, provided herein are isolated proteins comprising an amino acid sequence selected from Appendix B. In some embodiments, said isolated proteins may comprise at least 2 mutations relative to a SARS-CoV-2 Spike protein template sequence. In some such embodiments, the at least 2 mutations are located in the N-terminal Domain, Receptor Binding Domain, S2 subunit, or any combination thereof. In other embodiments, said isolated protein comprises at least 2 mutations relative to a SARS-CoV-2 Spike protein template sequence in each of the N-terminal Domain, Receptor Binding Domain, and S2 subunit. Such isolated proteins as are disclosed herein, comprising at least 2 mutations relative to a SARS-CoV-2 Spike protein template sequence, said SARS-CoV-2 Spike protein template sequence may be selected from Appendix A.

In other aspects of the invention, provided herein are isolated nucleic acids encoding the isolated proteins disclosed herein.

In additional aspects of the invention, provided herein are expression constructs comprising at least one isolated nucleic acid disclosed herein.

In further aspects of the invention, provided herein is a host cell comprising an expression construct disclosed herein.

In another aspect of the invention, provided herein are methods of producing an isolated protein, said methods may comprise expressing an isolated protein disclosed herein and at least partly purifying said isolated protein. Also provided herein are vaccine compositions comprising the isolated proteins disclosed herein and a pharmaceutically acceptable carrier. Additional aspects of the invention provided herein include vaccine compositions comprising the isolated nucleic acids disclosed herein. In some embodiments, the vaccine compositions provided herein, further comprise an adjuvant.

In yet further aspects of the invention, provided herein are methods of preventing or mitigating a SARS-CoV-2 infection in a subject, comprising administering to the subject a vaccine composition disclosed herein.

### EXAMPLES

### Example 1: Methods

1) Statistical modeling was employed to assign a “single-position VOC score” to each position in the viral proteome and genome that quantifies the propensity of that position to mutate (e.g., substituted, deleted, or as site for insertion) in future viral variants.

2) A machine-learning approach was employed to develop a classifier that uses the single-position VOC score as a feature to arrive at a binary classification for each viral amino acid position, indicating whether it is a “predicted future VOC position”.

3) The predicted future VOC positions were validated using test-cases as described herein. The single-position VOC score, and the classifier that uses single-position VOC score as a feature, were found to accurately predict positions that are mutated in future VOCs.

4) The predicted future VOC positions were distilled into specific “mutations of interest” that were considered in the design of potentially immunogenic sequences proposed herein. To do this the structural location of the predicted future VOC positions, and the nature of the mutations that have previously occurred at these positions, were considered.

5) Potentially immunogenic sequences were generated by placing the mutations of interest in “template” SARS-CoV-2 Spike protein sequences, for the protein in which predicted future VOC position occurs. The immunogenic sequences may be used in vaccine compositions, such as, and without limitation, by incorporation into appropriate vectors typically used in vaccination against viral pathogens (e.g., viral vectors or RNA delivery vehicles carrying RNA encoding the proposed immunogenic proteins).

Though the exemplified immunogenic peptides of the predicted VOCs described herein are focused on the spike protein, in some embodiments of the invention the workflow can be applied to generate potentially immunogenic sequences for other SARS-CoV-2 proteins, and that the same methodology can also be employed at the nucleotide level, to find nucleotide-level mutations predicted to occur in future VOCs.

### Example 2: Validation

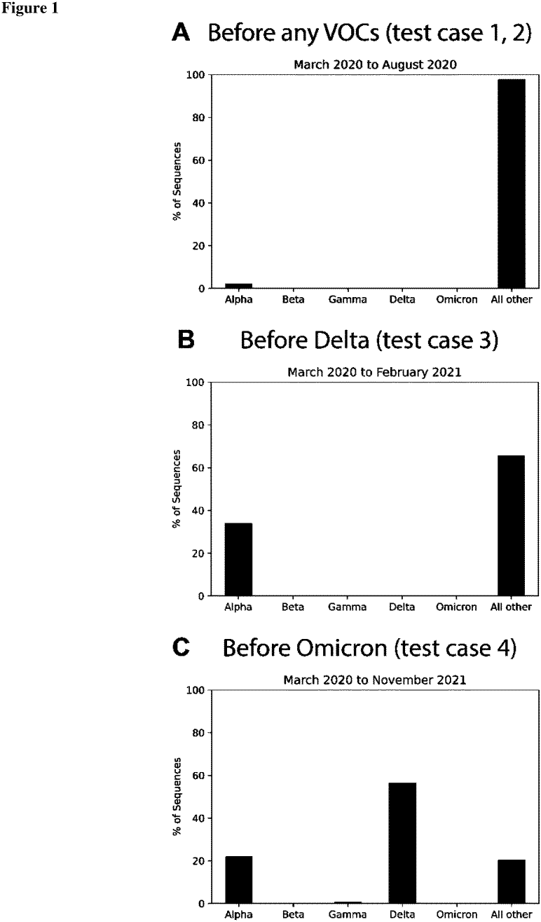

To assess the value of the single-position VOC scores or the prediction of positions that will be mutated in future VOCs, test scenarios were assessed wherein the various validation cases show the predictive capacity of classifiers based on the single-position VOC score, at various points in time. Specifically, the capacity of such classifiers to predict future VOC positions for VOCs that have not yet emerged was assessed in the time window used in each test case (FIG. 1). Although binary classifiers were used (i.e., a given position was either classified as a predicted future VOC position, or not), it should be noted that the single-position VOC score disclosed herein is a continuous quantity and that it can also be used to rank-order viral sequence positions, where higher ranked positions are predicted to mutate with increased likelihood.

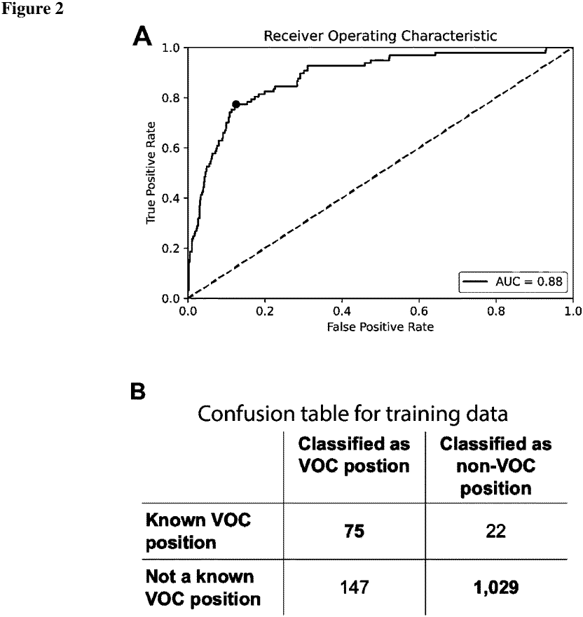

Test case 1: A single-position VOC score was used, calculated using only data from early in the COVID-19 pandemic (March 2020 - August 2020), prior to the emergence of VOCs (FIG. 1A), to train a classifier of known VOC future positions (for the Alpha, Beta, Gamma, and Delta VOCs). Whether the single-position VOC score correctly classifies positions based on whether they are mutated in known future VOCs was assessed. A ROC-curve was plotted and the AUC determined (FIG. 2A). The single-position VOC score was found to be a strong classifier (FIG. 2B), with an AUC of 0.88, a positive predictive value (PPV) of 0.34 and a false omission rate (FOR) of 0.02; indicating that positions with a high single-position VOC score are 16.1 times (PPV/FOR) more likely to be mutated in future VOCs than positions with a low single-position VOC score.

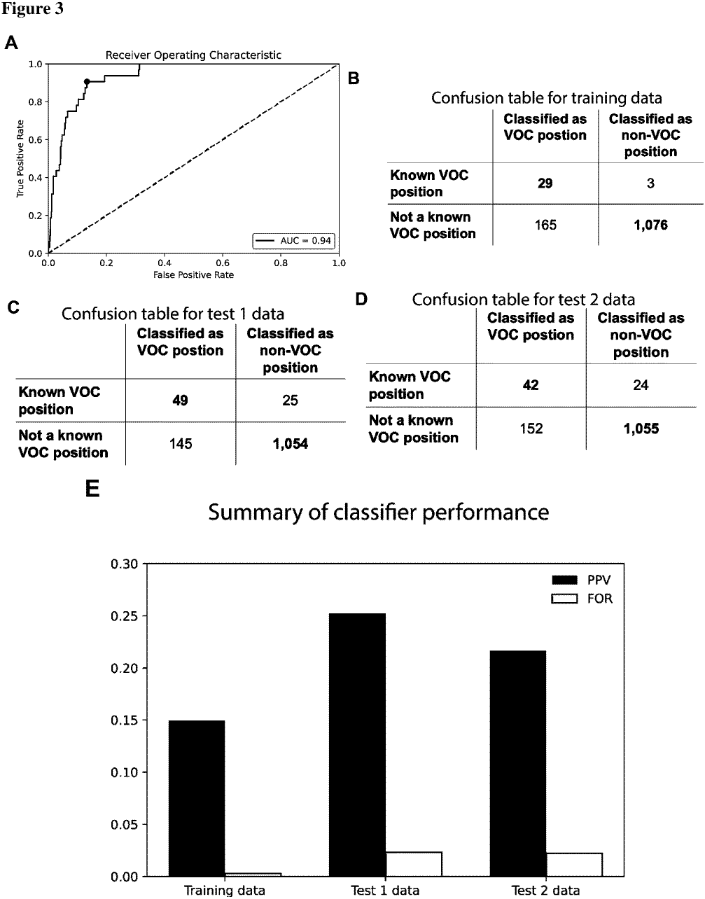

Test case 2: A single-position VOC score was used, calculated using only data from early in the pandemic (March 2020 - August 2020), prior to the emergence of VOCs (FIG. 1A), and trained a classifier to label known VOC positions for the Alpha, Beta, and Gamma VOCs (FIGS. 3A,B). The resulting classifier was used to predict future VOC positions and assess whether the predicted future VOC positions correctly capture Delta VOC positions that were not included in training the classifier. The classifier predicted Delta VOC positions with an accuracy of 0.87, and a 10.9-fold enrichment (PPV/FOR) of known Delta VOC positions in the predicted future VOC positions (FIG. 3C). Furthermore, the classifier predicted the subset of known Delta VOC positions that do not occur in any of the preceding VOCs with an accuracy of 0.86, and a 9.7-fold enrichment (PPV/FOR) in confirmed future VOC predictions in the predicted future VOC positions (FIG. 3D). Overall enrichment of confirmed VOC positions in the training and testing sets of positions are shown in FIG. 3E.

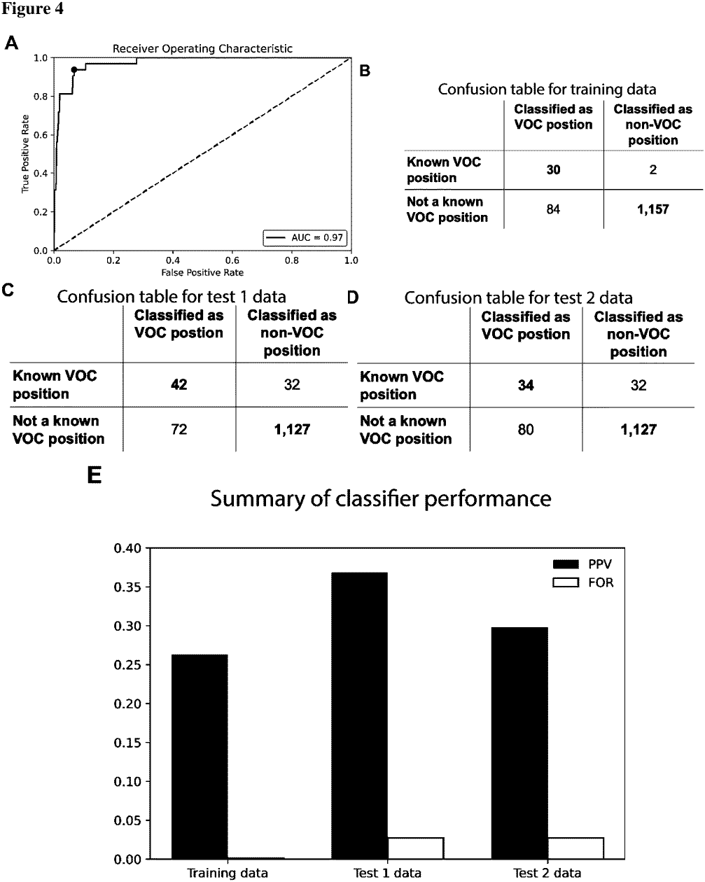

Test case 3: The single-position VOC score was used, calculated using only data from before emergence of the Delta variant (March 2020 - February 2021, FIG. 1B), and used to train a classifier to label known VOC positions for the Alpha, Beta, and Gamma VOCs (FIGS. 4A,B). The resulting classifier was used to predict future VOC positions and assess whether the predicted future VOC positions correctly captured Delta VOC positions that were not included in training the classifier. The classifier predicted known Delta VOC positions with an accuracy of 0.92, and a 13.3-fold enrichment (PPV/FOR) in confirmed Delta VOC positions in the predicted future VOC positions (FIG. 4C). Furthermore, the classifier predicted the subset of known Delta VOC positions that do not occur in any of the preceding VOCs with an accuracy of 0.91, and a 10.8-fold enrichment (PPV/FOR) in confirmed future VOC predictions in the predicted future VOC positions (FIG. 4D). Overall enrichment of confirmed VOC positions in the training and testing sets of positions are shown in FIG. 4E. This test-case demonstrates how additional training data can improve the prediction accuracy, compared to Test case 2.

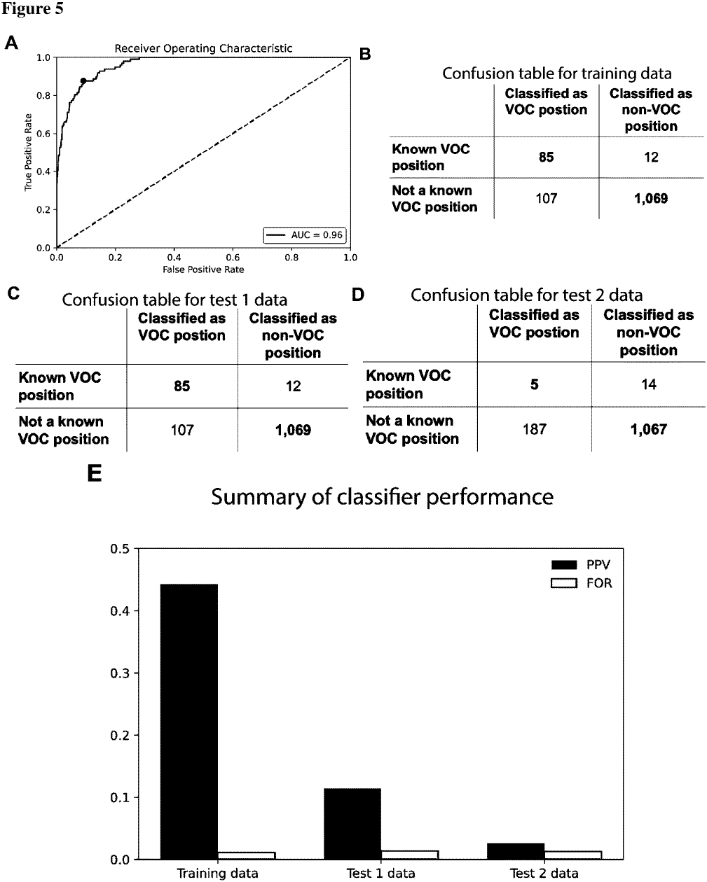

Test case 4: The single-position VOC score was used, calculated using data from before emergence of the Omicron variant (March 2020 - November 2021, FIG. 1C), and used to train a classifier to label known VOC positions for the Alpha, Beta, Gamma, and Delta VOCs (FIGS. 5A,B). The resulting classifier was used to predict future VOC positions and assess whether the predicted future VOC positions correctly captured Omicron VOC positions that were not included in training the classifier. The classifier predicted known Omicron VOC positions with an accuracy of 0.85, and a 8.3-fold enrichment (PPV/FOR) in confirmed Omicron VOC positions in the predicted future VOC positions (FIG. 5C). Furthermore, the classifier predicted the subset of known Omicron VOC positions that do not occur in any of the preceding VOCs with an accuracy of 0.84, and a 2.0-fold enrichment (PPV/FOR) in confirmed Omicron VOC positions in the predicted future VOC positions (FIG. 5D). Overall enrichment of confirmed VOC positions in the training and testing sets of positions are shown in FIG. 5E. The classifier used in this test case was also used in the forward prediction of the specific sequence described herein.

### Example 3: Determination of Specific Amino-acid Mutations Predicted to Occur in Future VOCs

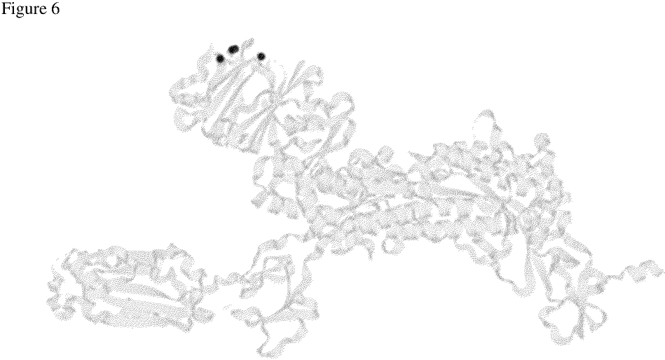

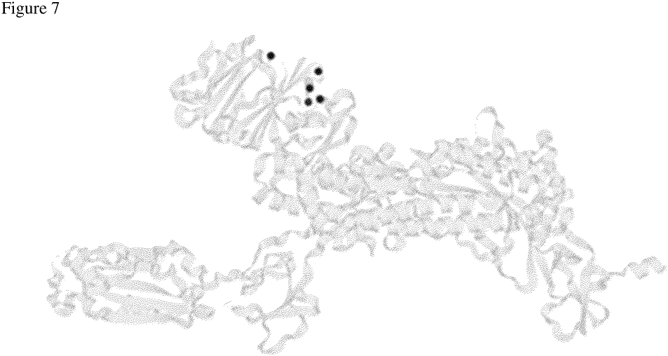

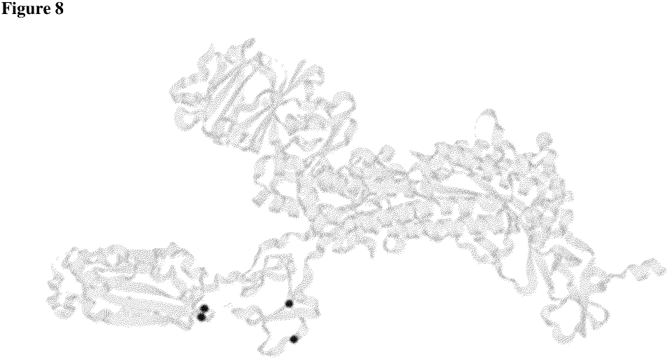

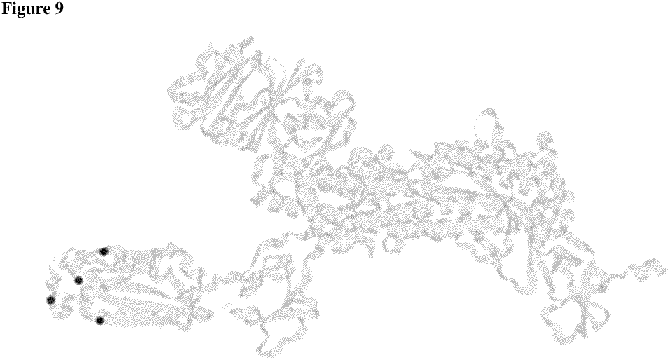

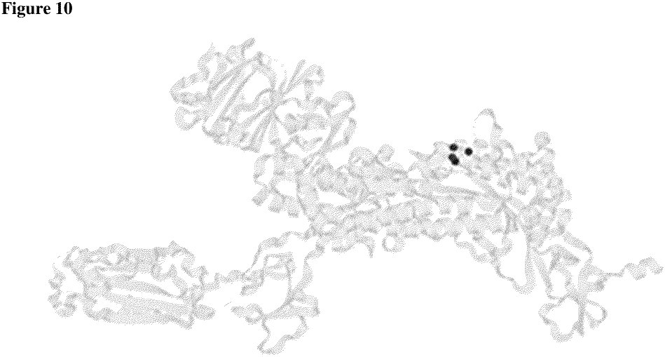

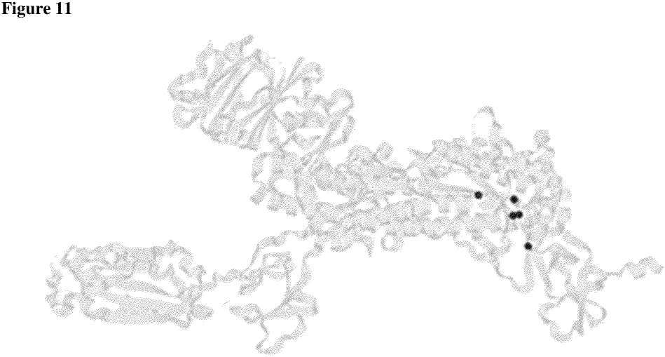

Based on sequencing data collected between March 2020 - November 2021, a total of 102 positions were predicted that are not yet part of current VOCs and that may mutate in future VOCs (FIG. 5). This list of positions was further refined using available structural data and data on the nature of mutations that have occurred in these positions to arrive at specific sets of 48 amino-acid mutations in 27 unique positions, clustered in six structurally co-localized sites on the Spike protein surface (FIGS. 6-11, Tables 1 and 2), that were predicted to occur in future VOCs, and that were used for designing immunogenic protein sequences. Following consideration of all possible permutations within the sites, the mutations disclosed herein resulted in 382 different sets of mutations, a tiny fraction of the total search space for future Spike protein variants, consisting of less than five mutations, if no prediction of future VOC positions was used, which equals approximately 10^20 sets of mutations.

Of the resulting 382 sets of mutations, sets with two or more mutations within a co-localized site are of particular interest, as multiple mutations within the same co-localized site are more likely to significantly impact inter-molecular interactions or local structure of the Spike protein. Each set of mutations described is restricted to a co-localized surface site (FIGS. 6-11), although it should be noted that such sets of mutations can also be combined in a modular fashion to arrive at additional potentially immunogenic protein sequences. The sites can be grouped as those that are likely to affect inter-molecular interactions (N-terminal Domain sites 1 and 2, and Receptor Binding Domain sites 3 and site 4, in the S1 subunit) and those that are likely to affect Spike protein structure (sites 5 and 6 in the S2 subunit).

Table 1: Structural sites of VOC mutations highlighted in the linear reference Spike protein sequence. All sequences below correspond to SEQ ID NO: 1.

Structural site 1 (N-terminal Domain)
1 MFVFLVLLPL VSSQCVNLTT RTQLPPAYTN SFTRGVYYPD KVFRSSVLHS TQDLFLPFFS NVTWFHAIHV SGTNGTKRFD NPVLPFNDGV YFASTENSNI 101 IRGWIFGTTL DSKTGSLLIV NNATNVVIKV CEFQFCNDPF L GVYYHKNNK SWMESEFRVY SSANNCTFEY VSQPFLMDLE GKQGNFKNLR EFVFKNIDGY 201 FKIYSKHTPI NLVRDLPQGF SALEPLVDLP IGINITRFQT LLALHR SYLT PGDSSSGWTA G AAAYYVGYL QPRTFLLKYN ENGTITDAVD CALDPLSETK 301 CTLKSFTVEK GIYQTSNFRV QPTESIVRFP NITNLCPFGE VFNATRFASV YAWNRKRISN CVADYSVLYN SASFSTFKCY GVSPTKLNDL CFTNVYADSF 401 VIRGDEVRQI APGQTGKIAD YNYKLPDDFT GCVIAWNSNN LDSKVGGNYN YLYRLFRKSN LKPFERDIST EIYQAGSTPC NGVEGFNCYF PLGSYGFQFT 501 NGVGYQPYRV VVLSFELLHA PATVCGPKKS TNLVKNKCVN FNFNGLTGTG VLTESNKKFL PFQQFGRDIA DTTDAVRDPQ TLEILDITPC SFGGVSVITP 601 GTNTSNQVAV LYQDNVCTEV PVAIHADQLT PTWRVYSTGS NVFQTRAGCL IGAEHVNNSY ECDIPIGAGI CASYQTQTNS PRRARSVASQ SIIAYTMSLG 701 AENSVAYSNN SIAIPTNFTI SVTTEILPVS MTKTSVDCTM YICGDSTECS NLLQYGSFC TQLNRALTGI AVEQDKNTQE VFAQVKQIYK TPPIKDFGGF 801 NFSQILPDPS KPSKRSFIED LLFNKVTLAD AGFIKQYGDC LGDIAARDLI CAQKFNGLTV LPPLLTDEMI AQYTSALLAG TITSGWTFGA GAALQIPFAM 901 QMAYRFNGIG VTQNVLYENQ KLIANQFNSA IGKIQDSLSS TASALGKLQD VVNQNAQALN TLVKQLSSNF GAISSVLNDI LSRLDKVEAE VQIDRLITGR 1001 LQSLQTYVTQ QLIRAAEIRA SANLAATKMS ECVLGQSKRV DFCGKGYHLM SFPQSAPHGV VFLHVTYVPA QEKMFTTAFA ICHDGKAHFP REGVFVSNGT 1101 HWFVTQRNFY EPQIITTDNT FVSGNCDVVI GIVNNTVYDP LQPELDSFKE ELDKYFKNHT SPDIVDLGDIS GINASVVNIQ KEIDRLNEVA KNLNESLIDL 1201 QELGKYEQYI KWPWYIWLGF IAGLIAIVMV TIMLCCMTSC CSCLKGCCSC GSCCKFDEDD SEPVLKGVKL HYT
Structural site 2 N-terminal Domain)
1 MFVFLVLLPL VSSQCVNLTT RTQLPPAYTN SFTRGVYYPD KVFRSSVLHS TQDLFLPFFS NVTWFHAIHV SGTNGTKRFD NPVLPFNDGV YFASTEKSNI 101 IRGWIFGTTL DSKTQSLLIV NNATNVVIKV CEFQFCNDPF LGVYYHKNNK SWMESEFRVY SSANNCTFEY VSQPFL MDLE GKQGNFKNLR EFVFKNIDGY 201 FKIYSKHTPI NLVR DL PQGF S ALEPLVDLP IGINITRFQT LLALHRSYLT PGDSSSGWTA GAAAYYVGYL QPRTFLLKYN ENGTITDAVD CALDPLSETK 301 CTLKSFTVEK GIYQTSNFRV QPTESIVRFP NITNLCPFGE VFNATRFASV YAWNRKRISN CVADYSVLYN SASFSTFKCY GVSPTKLNDL CFTNVYADSF 401 VIRGDEVRQI APGQTGKIAD YNYKLPDDFT GCVIAWNSNN LDSKVGGMYN YLYRLFRKSN LKPFERDIST EIYQAGSTPC NGVEGFNCYF PLQSYGFQPT 501 NGVGYQPYRV VVLSFELLHA PATVCGPKKS TNLVKNKCVN FNFNGLTGTG VLTESNKKFL PFQQFGRDIA DTTDAVRDPQ TLEILDITPC SFGGVSVITP 601 GTNTSNQVAV LYQDVNCTEV PVAIHADQLT PTWRVYSTGS NVFQTRAGCL IGAEHVNNSY ECDIPIGAGI CASYQTQTNS PRRARSVASQ SIIAYTMSLG 701 AENSVAYSNN SIAIPTNFTI SVTTEILPVS MTKTSVDCTM YICGDSTECS NLLLQYGSFC TQLNRALTGI AVEQDKNTQE VFAQVKQIYK TPPRIKDFGGF 801 NFSQILPDPS KPSKRSFIED LLFNKVTLAD AGFIKQYGDC LGDIAARDLI CAQKFMGLTV LPPLLTDEMI AQYTSALLAG TITSGWFGA GAALQIPFAM 901 QMAYRFNGIG VTQNVLYENQ KLIANQFNSA IGKIQDSLSS TASALGKLQD VVNQNAQALN TLVKQLSSNF GAISSVLNDI LSRLDKVEAE VQIDRLITGR 1001 LQSLQTYVTQ QLIRAAEIRA SANLAATKMS ECVLGQSKRV DFCGKGYHLM SFPQSAPHGV VFLHVTYVPA QEKNFTTAPA ICHDGKAHFP REGVFVSNGT 1101 HWFVTQRNFY EPQIITTDNT FVSGNCDVVI GIVNNTVYDP LQPELDSFKE ELDKYFXNHT SPDVDLGDIS GINASVVNIQ KEIDRLNEVA KNLNESLIDL 1201 QELGKYEQYI KWPWYIWLGF IAGLIAIVMV TIMLCCMTSC CSCLKGCCSC GSCCKFDEDD SEPVLKGVKL HYT
Structural site 3 (Receptor Binding Domain)
1 MFVFLVLLPL VSSQCVNLTT RTQLPPAYTN SFTRGVYYPD KVFRSSVLHS TQDLFLPFFS NVTWFHAIHV SGINGTKRFD NPVLPFNDGV YFASTEKSNI 101 IRGWIFGTTL DSKTQSLLIV NNATNVVIKV CEFQFCNDPF LGVYYHKNNK SWMESEFRVY SSANNCTFEY VSQPFLMDLE GKQGNFKNLR EFVFKNIDGY 201 FKIYSKHTPI NLVRDLPQGF SALEPLVDLP IGINITRFQT LLALHRSYLT PGDSSSGWTA GAAAYYVGYL QPRTFLLKYN ENGTITDAVD CALDPLSETK 301 CTLKSFTVEK GIYQTSNFRV QPTESIVRFP NITNLCPFGE VFNATRFASV YAWNRKRISM CVADYSVLYN SASFSTFKCY GVSPTKLNDL CFTNVYADSF 401 VIRGDEVRQI APGQTGKIAD YNYKLPDDFT GCVIAWNSNN LDSKVGGNYN YLYRLFRKSN LKPFERDIST EIYQAGSTPC NGVEGFNCYF PLQSYGFQPT 501 NGVGYQPYRV VVLSFELLHA PA TVCGPKKS THLVKNKCVN FNFNGLTGTG VLTE SNKK FL PFQQFGRDIA DTTDAVRDPQ TLEILDITPC SFGGVSVITP 601 GTNTSNQVAV LYQDVNCTEV PVAIHADQLT PTWRVYSTGS NVFQTRAGCL IGAEHVNNSY ECDIPIGAGI CASYQTQTNS PRRARSVASQ SIIAYTMSLG 701 AENSVAYSNN SIAIPTNFTI SVTTEILPVS MTKTSVDCTM YICGDSTECS NLLLQYGSFC TQLNRALTGI AVEQDKNTQE VFAQVKQIYK TPPIKDFGGF 801 NFSQILPDPS KPSKRSFIED LLFNKVTLAD AGFIKQYGDC LGDIAARDLI CAQKFNGLTV LPPLLTDEMI AQYTSALLAG TITSGWTFGA GAARLQIPFAM 901 QMAYRFNGIG VTQNVLYENQ KLIANQFNSA IGKIQDSLSS TASALGKLQD VVNQNAQALN TLVKQLSSNF GAISSVLNDI LSRLDKVEAE VQIDRLITGR 1001 LQSLQTYVTQ QLIRAAEIRA SANLAATKMS ECVLGQSKRV DFCGKGYHLM SFPQSAPHGV VFLHVTYVPA QEKNFTTAPA ICHDGKAHFP REGVFVSNGT 1101 HWFVTQRNFY EPQIITTDNT FVSGNCDVVI GIVNNTVYDP LQPELDSFKE ELDEYFKNHT SPDVDLGDIS GINASVVNIQ KEIDRLNEVA KNLNESLIDL 1201 QELGKYEQYI KWPWYIWLGF IAGLIAIVMV TIMLCCMTSC CSCLKGCCSC GSCCKFDEDD SEPVLKGVKL HYT
Structural site 4 (Receptor Binding Domain)
1 MFVFLVLLPL VSSQCVNLTT RTQLFPAYTN SFTRGVYYPD XYFRSSVLHS TQDLFLPFFS NVTWFHAIHV SGTNGTKRFD NPVLPFNDGV YFASTEKSNI 101 IRGWIFGTTL DSKTQSLLIV NNATNVVIKV CEFQFCNDPF LGVYYHKNNK SWMESEFRVY SSANNCTFEY VSQPFLMDLE GKQGNFKNLP EFVFKNIDGY 201 FKIYSKHTPI NLVRDLPQGF SALEPLVDLP IGINITRFQT LLALHRSYLT PGDSSSGWTA GAAAYYVGYL QPRTFLLKYN ENGTITDAVD CALDPLSETK 301 CTLKSFTVEK GIYQTSNFRV QPTESIVRFP NITNLCPFGE VFNATR FASV YAWNRKRISN CVADYSVLYN SASFSTFKCY GVSPTKLNDL CFTNVYADSF 401 VIRGDEVRQI APGQ TGKIAD YNYKLPDDFT GCVIAWNSN N LDSKVGGNYN YLYRLFRKSN LKPFERDIST EIYQAGSTPC NGVEGFNCYF PLQS YGFQPT 501 NGVGYQPYRV VVLSFELLHA PATVCGPKKS TNLVKNKCVN FNFNGLTGTG VLTESNKKFL PFQQFGRDIA DTTDAVRDPQ TLEILDITPC SFGGVSVITP 601 GTNTSNQVAV LYQDVNCTEV PVAIHADQLT PTWRVYSTGS NVFQTFAGCL IGAEHVNNSY ECDIPIGAGI CASYQTQTNS PRRARSVASQ SIIAYTMSLG 701 AENSVAYSNN SIAIPTNFTI SVTTEILPVS MTKTSVDCTM YICGDSTECS NLLLQYGSFC TQLNRALTGI AVEQDKNTQE VFAQVKQIYK TPPIKDFGGF 801 NFSQILPDPS KPSKRSFIED LLFNKVTLAD AGFIKQYGDC LGDIAARDLI CAQKFNGLTV LPPLLTDEMI AQYTSALLAG TITSGWTFGA GAALQIPFAM 901 QMAYRFNGIG VTQNVLYENQ KLIANQFNSA IGKIQDSLSS TASALGKLQD VVNQNAQALN TLVKQLSSNF GAISSVLNDI LSRLDKVEAE VQIDRLITGR 1001 LQSLQTYVTQ QLIRAAEIRA SANLAATKMS ECVLGQSKRV DFCGKGYHLM SFPQSAPHGV VFLHVTYVPA QEKNFTTAPA ICHDGKAHFP REGVFVSNGT 1101 HWFVTQRNFY EPQIITTDNT FVSGNCDVVI GIVNNTVYDP LQPELDSFKE ELDKYFKNHT SPDVDLGDIS GINASVVNIQ KEIDRLNEVA KNLNESLIDL 1201 QELGKYEQYI KWPWYIWLGF IAGLIAIVMV TIMLCCMTSC CSCLKGCCSC GSCCKFDEDD SEPVLKGVKL HYT
Structural site 5 (Receptor S2 Subunit)
1 MFVFLVLLPL VSSQCVNLTT PTQLPPAYTN SFTRGVYYPD KVFRSSVLHS TQDLFLPFFS NVTWFHAIHV SGTNGTKRFD NPVLPFNDGV YFASTEKSNI 101 IRGWIFGTTL DSKTQSLLIV NNATNVVIKV CEFQFCNDPF LGVYYHKNNK SWMESEFRVY SSANNCTFEY VSQPFLMDLE GKQGNFKNLR EFVFKNIDGY 201 FKIYSKHTPI NLVRDLPQGF SALEPLVDLP IGINITRFQT LLALHRSYLT PGDSSSGWTA GAAAYYVGYL QPRTFLLKYN ENGTITDAVD CALDPLSETK 301 CTLKSFTVEK GIYQTSNFRV QPTESIVRFP NITNLCPFGE VFNATRFASV YAWNRKRISN CVADYSVLYN SASFSTFKCY GVSPTKLNDL CFTNVYADSF 401 VIRGDEVRQI APGQTGKIAD YNYKLPDDFT GCVIAWNSNN LDSKVGGNYN YLYRLFRKSN LKPFERDIST EIYQAGSTPC NGVEGFNCYF PLQSYGFQPT 501 NGVGYQPYRV VVLSFELLHA PATVCGPKKS TNLVKNKCVN FNFNGLTGTG VLTESNKKFL PFQQFGRDIA DTTDAVRDPQ TLEILDITPC SFGGVSVITP 601 GTNTSNQVAV LYQDVNCTEV PVAIHADQLT PTWRVYSTGS NVFQTRAGCL IGAEHVNNSY ECDIPIGAGI CASYQTQTNS PRRARSVASQ SIIAYTMSLG 701 AENSVAYSNN SIAIPTNFTI SVTTEILPVS MTKTSVDCTM YICGDSTECS NLLLQYGSFC TQLNRALTGI AVEQDKNTQE VFAQVKQIYK TPPIKDFGGF 801 NFSQILPDPS KPSKRSFIED LL FNKVTLAD AGFIKQYGDC LGDIAARDLI CAQKFNGLTV LPPLLTDEMI AQYTSALLAG TITSGWTFGA GAALQIPFAM 901 QMAYRFNGIG VTQNVLYENQ KLIANQFNSA IGKIQD SLS S TASALGKLQD VVNQNAQALN TLVKQLSSNF GAISSVLNDI LSRLDKVEAE VQIDRLITGR 1001 LQSLQTYVTQ QLIRAAEIRA SANLAATKMS ECVLGQSKRV DFCGKGYHLM SFPQSAPHGV VFLHVTYVPA QEKNFTTAPA ICHDGKAHFP REGVFVSNGT 1101 HWFVTQRNFY EPQIITTDNT FVSGNCDVVI GIVNNTVYDP LQPELDSFKE ELDKYFKNHT SPDVDLGDIS GINASVVNIQ KEIDRLNEVA KNLNESLIDL 1201 QELGKYEQYI KWPWYIWLGF IAGLIAIVMV TIMLCCMTSC CSCLKGCCSC GSCCKFDEDD SEPVLKGVKL HYT
Structural site 6 (Receptor S2 Subunit)
1 MFVFLVLLPL VSSQCVNLTT RTQLPPAYTN SFTRGVYYPD KVFRSSVLHS TQDLFLPFFS NVTWFHAIHV SGTNGTKRFD NPVLPNDGV YFASTEKSNI 101 IRGWIFGTTL DSKTQSLLIV NNATNVVIKV CEFQFCNDPF LGVYYHKNNK SWMESEFRVY SSANNCTFEY VSQPFLMDLE GKQGNFKNLR EFVFKNIDGY 201 FKIYSKHTPI NLVRDLPQGF SALEPLVDLP IGINITRFQT LLALHRSYLT PGDSSSGWTA GAAAYYVGYL QPRTFLLKYN ENGTITDAVD CALDPLSETK 301 CTLKESFTVEK GIYQTSNFRV QPTESIVRFP NITNLCPFGE VFNATRFASV YAWNRKRISN CVADYSVLYN SASFSTFKCY GVSPTKLNDL CFTNVYADSF 401 VIRGDEVRQI APGQTGKIAD YNYKLPDDFT GCVIAWNSNN LDSKVGGNYN YLYRLFRKSN LKPFERDIST EIYQAGSTPC NGVEGFNCYF PLQSYGFQPT 501 NGVGYQPYRV VVLSFELLHA PATVCGPKKS TNLVKNKCVN FNFNGLTGTG VLTESNKKFL PFQQFGRDIA DTTDAVRDPQ TLEILDITPC SFGGVSVITP 601 GTNTSNQVAV LYQDVNCTEV PVAIHADQLT PTWRVYSTGS NVFQTRAGCL IGAEHVNNSY ECDIPIGAGI CASYQTQTNS PPRARSVASQ SIIAYTMSLG 701 AENSVAYSNN SIAIPTNFT I SVT TEILPVS MTKTSVDCTM YICGDSTECS NLLLQYGSFC TQLNRALTGI AVEQDKNTQE VFAQVKQIYK TPPIKDFGGF 801 NFSQILPDPS KPSKRSFIED LLFNKVTLAD AGFIKQYGDC LGDIAARDLI CAQKFNGLTV LPPLLTDEMI AQYTSALLAG TITSGWTFGA GAALQIPFAM 901 QMAYRFNGIG VTQNVLYENQ KLIANQFNSA IGKIQDSLSS TASALGKLQD VVNQNAQALN TLVKQLSSNF GAISSVLNDI LSRLDKVEAE VQIDRLITGR 1001 LQSLQTYVTQ QLIRAAEIRA SANLAATKMS ECVLGQSKRV DFCGKGYHLM SFPQSAPHGV VFLHVTYVPA Q EKN FTTAPA ICHDGKAHFP REGVFVSNGT 1101 HWFVTQRNFY EPQIITTDNT FVSGNCDVVI GIVNNTVYDP LQPELDSFKE ELDKYFXNHT SPDVDLGDIS GINASVVNIQ KEIDRLNEVA KNLNESLIDL 1201 QELGKYEQYI KWPWYIWLGF IAGLIAIVMV TIMLCCMTSC CSCLKGCCSC GSCCKFDEDD SEPVLKGVKL HYT

Table 2: Overview of predicted mutations grouped by site.

| Site Number | Amino Acids Contained | Predicted Mutations |
|---|---|---|
| 1 | L141 H245 R246 G261 | F, deletion Y I, deletion V, C, R, D |
| 2 | L176 I210 R214 L216 S221 | F T L F L |
| 3 | A520 A522 E554 K558 | S S, V, P G, Q N |
| 4 | R346 Q414 N439 F490 S494 | S, I K K S, L P, L |
| 5 | L822 D936 L938 S939 | F Y, N, H F F |
| 6 | T719 T723 A1070 Q1071 N1074 | I I S, V H, L S, D |

### Example 4: Design of the Potential Immunogenic Protein Sequences

In order to design immunogenic protein sequences based on the mutations that were predicted to occur in future VOCs, templates based on three prevalent versions of the SARS-CoV-2 Spike protein (reference strain, 21 Delta variant, and Omicron variant), as well as alternate versions of these templates including stabilizing 2P mutations, 22-24 were used, wherein the mutations were combinatorially introduced into said templates. Here, only sequences with at least one of the predicted future VOC mutations were considered. Specifically, the following steps were used to design immunogenic protein sequences starting from the predicted sets of future VOC mutations:

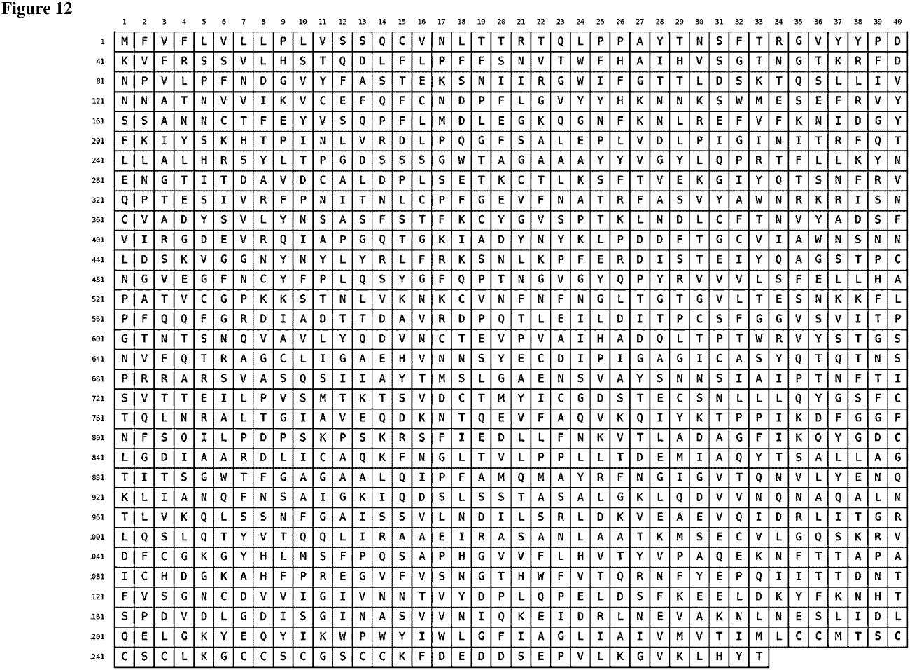

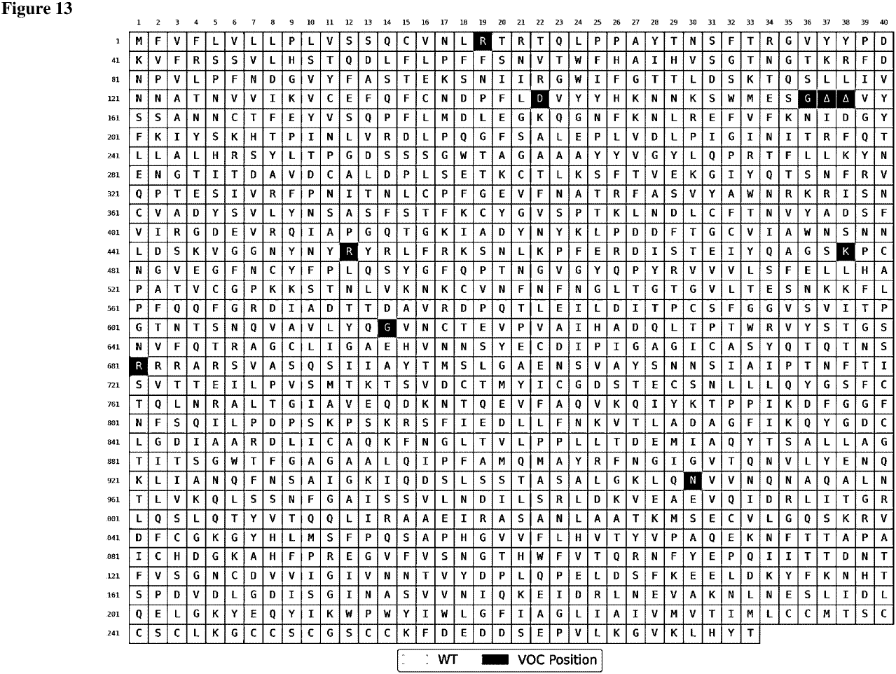

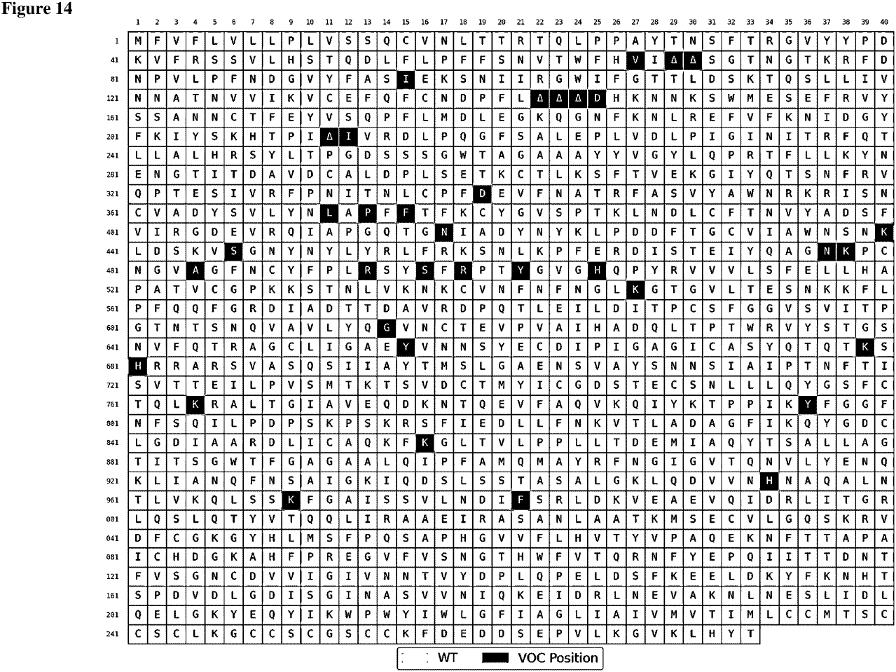

1) Design of three template Spike protein sequences; one based on the reference SARS-CoV-2 strain (GenBank: QHD43416.1) (FIG. 12), one based on the Delta variant (FIG. 13), and one based on the Omicron variant (FIG. 14), that serve as a template sequence into which the additional predicted mutations were introduced (see Appendix A). These templates were designed by starting from the reference Spike protein and introducing core mutations25 of the Delta and Omicron variants respectively. For each template, a version with the stabilizing 2P mutations added was also generated, yielding a total of six template Spike protein sequences.

2) Introduction of predicted future VOC mutations into the template sequences, one structural site at a time, considering all possible combinations of at least one predicted future VOC mutations. This resulted in the following number of unique sequences per structural site, and a total of 2,292 unique potential immunogenic protein sequences (see Appendix B):
a) Structural site 1: 59 sequences per template x 6 templates = 354 sequences
b) Structural site 2: 31 sequences per template x 6 templates = 186 sequences
c) Structural site 3: 47 sequences per template x 6 templates = 282 sequences
d) Structural site 4: 107 sequences per template x 6 templates = 642 sequences
e) Structural site 5: 31 sequences per template x 6 templates = 186 sequences
f) Structural site 6: 107 sequences per template x 6 templates = 642 sequences

The designed immunogenic protein sequences, or combinations thereof, could then be inserted in viral vectors or encoded by mRNA, or otherwise administered as a vaccine by methods known in the art, e.g., inducing the production of the immunogenic protein sequences by the cells of the vaccinated individuals, administered via methods such as recombinant vaccines, peptide/subunit vaccines, and the like. In some embodiments the same steps employed here are used to design potentially immunogenic sequences for other SARS-CoV-2 proteins, and the related nucleotide sequences.

### Example 5: Use of the Designed Immunogenic Protein Sequences for COVID-19 Vaccines Against Predicted Future Variants

In order to induce an immune response against future SARS-CoV-2 variants of concern, the immunogenic proteins disclosed herein can be used to compose vaccines. Vaccines that use viral proteins work by inputting a virus-specific protein into an individual, inducing an immune response within the vaccine recipient.26 Since the immunogenic protein is foreign to the body, the subject’s immune system produces antibodies to remove it. These antibodies should therefore protect the patient if they become infected with the virus.

Without being bound by theory and solely for the purpose of exemplification, vaccine strategies include delivery of nucleic acids encoding the immunogenic protein to cells in the vaccine recipient, thereby inducing the cells of the recipient to produce said immunogenic protein, e.g., messenger RNA (mRNA) and viral vector vaccines. For Example, mRNA vaccines put mRNA encoding the immunogenic protein in lipids before injecting them into the body.27,28 The mRNA will then enter cells and cause them to produce the immunogenic protein. Vaccines may also comprise viral vectors that deliver the nucleic acid encoding the immunogenic protein into cells of the vaccine recipient inducing them to produce the encoded protein.29,30 The development of such mRNA or viral vector vaccines could be greatly accelerated using the methods disclosed herein, and particularly for the proactive development of COVID-19 vaccines using the predicted immunogenic protein sequences disclosed herein.

On the other hand, subunit, recombinant, polysaccharide, and conjugate vaccines all use parts of the virus itself to trigger an immune response. For example, while mRNA and viral vector vaccines for COVID-19 use either DNA or mRNA to cause the body to make a specific S protein, a subunit vaccine for COVID-19 would use the S protein itself, e.g., directly administering the immunogenic protein, or compositions thereof, to the subject.⁶ The design of such subunit (peptide) vaccines for future COVID-19 variants may comprise the potential immunogenic proteins described herein, or fragments thereof to elicit an immune response in the vaccine recipient, thus providing protection against VOCs.

### REFERENCES

1. COVID-19 Map - Johns Hopkins Coronavirus Resource Center.https://coronavirus.jhu.edu/map.html.

2. Pawlowski, C.et al.FDA-authorized COVID-19 vaccines are effective per real-world evidence synthesized across a multi-state health system.bioRxiv(2021) doi:10.1101/2021.02.15.21251623.

3. Thompson, M. G. Interim Estimates of Vaccine Effectiveness of BNT162b2 and mRNA-1273 COVID-19 Vaccines in Preventing SARS-CoV-2 Infection Among Health Care Personnel, First Responders, and Other Essential and Frontline Workers - Eight U.S. Locations, December 2020-March 2021.MMWR Morb. Mortal. Wkly. Rep.70, (2021).

4. Polack, F. P.et al.Safety and Efficacy of the BNT162b2 mRNA Covid-19 Vaccine.N. Engl. J. Med.383, 2603-2615 (2020).

5. Baden, L. R.et al.Efficacy and Safety of the mRNA-1273 SARS-CoV-2 Vaccine.N. Engl. J. Med.384, 403-416 (2021).

6. Thompson, M. G.et al.Effectiveness of Covid-19 Vaccines in Ambulatory and Inpatient Care Settings.N. Engl. J. Med.(2021) doi:10.1056/NEJMoa2110362.

7. Lopez Bernal, J.et al.Effectiveness of Covid-19 Vaccines against the B.1.617.2 (Delta) Variant.N. Engl. J. Med.385, 585-594 (2021).

8. Bar-On, Y. M.et al.BNT162b2 vaccine booster dose protection: A nationwide study from Israel.bioRxiv(2021) doi:10.1101/2021.08.27.21262679.

9. Barda, N.et al.Effectiveness of a third dose of the BNT162b2 mRNA COVID-19 vaccine for preventing severe outcomes in Israel: an observational study.Lancet398, 2093-2100 (2021).

10. Bar-On, Y. M.et al.Protection of BNT162b2 Vaccine Booster against Covid-19 in Israel.N. Engl. J. Med.385, 1393-1400 (2021).

11. Arbel, R.et al.BNT162b2 Vaccine Booster and Mortality Due to Covid-19.N. Engl. J. Med.385, 2413-2420 (2021).

12. Puranik, A.et al.Durability analysis of the highly effective BNT162b2 vaccine against COVID-19.bioRxiv(2021) doi:10.1101/2021.09.04.21263115.

13. Israel, A.et al.Elapsed time since BNT162b2 vaccine and risk of SARS-CoV-2 infection in a large cohort.medRxiv(2021) doi:10.1101/2021.08.03.21261496.

14. Classification of Omicron (B.1.1.529): SARS-CoV-2 Variant of Concern.https://www.who.int/news/item/2Jun. 11, 2021-classification-of-omicron-(b.1.1.529)-sars-cov-2-variant-of-concern.

15. Gobeil, S. M.-C.et al.Effect of natural mutations of SARS-CoV-2 on spike structure, conformation, and antigenicity.Science(2021) doi:10.1126/science.abi6226.

16. Wang, P.et al.Antibody resistance of SARS-CoV-2 variants B.1.351 and B.1.1.7.Nature(2021) doi:10.1038/s41586-021-03398-2.

17. Shen, X.et al.Neutralization of SARS-CoV-2 Variants B.1.429 and B.1.351.N. Engl. J. Med.(2021) doi:10.1056/NEJMc2103740.

18. McCarthy, K. R.et al.Recurrent deletions in the SARS-CoV-2 spike glycoprotein drive antibody escape.Science371, 1139-1142 (2021).

19. Collier, D. A.et al.Sensitivity of SARS-CoV-2 B.1.1.7 to mRNA vaccine-elicited antibodies.Nature593, 136-141 (2021).

20. Harvey, W. T.et al.SARS-CoV-2 variants, spike mutations and immune escape.Nat. Rev. Microbiol.(2021) doi:10.1038/s41579-021-00573-0.

21. Wu, F.et al.A new coronavirus associated with human respiratory disease in China.Nature579, 265-269 (2020).

22. Pallesen, J.et al.Immunogenicity and structures of a rationally designed prefusion MERS-CoV spike antigen.Proc. Natl. Acad. Sci. U. S. A.114, E7348-E7357 (2017).

23. Kirchdoerfer, R. N.et al.Stabilized coronavirus spikes are resistant to conformational changes induced by receptor recognition or proteolysis.Sci. Rep.8, 15701 (2018).

24. Wrapp, D.et al.Cryo-EM structure of the 2019-nCoV spike in the prefusion conformation.Science367, 1260-1263 (2020).

25. Suratekar, R.et al.High diversity in Delta variant across countries revealed via genome-wide analysis of SARS-CoV-2 beyond the Spike protein.bioRxiv2021.09.01.458647 (2021) doi:10.1101/2021.09.01.458647.

26. Office of Infectious Disease & HIV/AIDS Policy (OIDP). Vaccine Types.HHS.govhttps://www.hhs.gov/immunization/basics/types/index.html(2021).

27. mRNA Vaccines.BioNTechhttps://www.biontech.de.

28. CDC. Understanding mRNA COVID-19 Vaccines.Centers for Disease Control and Preventionhttps://www.cdc.gov/coronavirus/2019-ncov/vaccines/different-vaccines/mrna.html(2022).

29. Bolhassani, A. & Yazdi, S. R. DNA immunization as an efficient strategy for vaccination.Avicenna J. Med. Biotechnol.1, 71-88 (2009).

30. CDC. Understanding Viral Vector COVID-19 Vaccines.Centers for Disease Control and Prevention,www.cdc.gov/coronavirus/2019-ncov/vaccines/different-vaccines/viralvector.html(2021).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated protein, comprising an amino acid sequence selected from Appendix B.

2. The isolated protein of claim 1, wherein said isolated protein comprises at least 2 mutations relative to a SARS-CoV-2 Spike protein template sequence, wherein the at least 2 mutations are located in the N-terminal Domain, Receptor Binding Domain, S2 subunit, or any combination thereof.

3. The isolated protein of claim 1, wherein said isolated protein comprises at least 2 mutations relative to a SARS-CoV-2 Spike protein template sequence in each of the N-terminal Domain, Receptor Binding Domain, S2 subunit, or any combination thereof.

4. The isolated protein of claim 1,wherein the SARS-CoV-2 Spike protein template sequence is selected from Appendix A.

5. An isolated nucleic acid encoding the isolated protein of any preceding claim.

6. An expression construct comprising the isolated nucleic acid of claim 5.

7. A host cell comprising the expression construct of claim 6.

8. A method of producing an isolated protein, said method comprising expressing the isolated protein in the host cell of claim 7 and at least partly purifying the isolated protein.

9. A vaccine composition comprising the isolated protein of claim 1 and a pharmaceutically acceptable carrier.

10. A vaccine composition comprising the isolated nucleic acid of claim 5.

11. The vaccine composition of claim 9, further comprising an adjuvant.

12. A method of preventing or mitigating a SARS-CoV-2 infection in a subject, comprising administering to the subject the vaccine composition of claim 9.


SEQUENCE LISTING (3 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1271 aa, Severe acute respiratory syndrome coronavirus 2
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1267 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT